(19) United States
(12) Patent Application Publication
(10) Pub. No.: US 2024/0343661 A1
(43) Pub. Date: Oct. 17, 2024

(54) CATALYST AND PROCESS FOR HYDROGENATION OF OLEFINS

(71) Applicant: Evonik Oxeno GmbH & Co. KG, Marl (DE)

(72) Inventors: Ralf Meier, Sendenhorst (DE); Guido Stochniol, Haltern am See (DE); Stephan Peitz, Oer-Erkenschwick (DE); Armin Matthias Rix, Marl (DE)

(73) Assignee: Evonik Oxeno GmbH & Co. KG, Marl (DE)

(21) Appl. No.: 18/631,125

(22) Filed: Apr. 10, 2024

(30) Foreign Application Priority Data
Apr. 11, 2023 (EP) 23167262.7

(51) Int. Cl. C07C 5/03

(52) U.S. Cl. CPC C07C 5/03; C07C 2521/12; C07C 2523/42

(57) ABSTRACT

A catalyst for the total hydrogenation of an olefin-containing hydrocarbon stream can be made. The catalyst has a support material and platinum as a catalytically active metal. A process for the total hydrogenation of an olefin-containing hydrocarbon stream in at least one reactor with addition of hydrogen and using the catalyst can be performed.

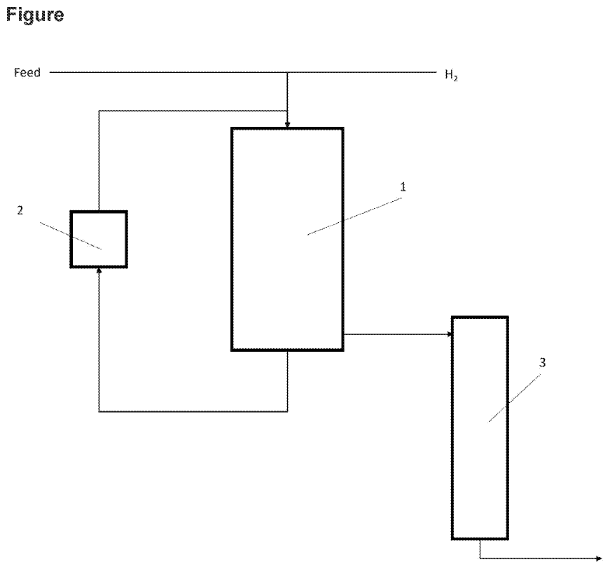

### CROSS-REFERENCE TO RELATED APPLICATIONS

This application claims priority to European Application No. 23167262.7 filed on Apr. 11, 2023, the content of which is hereby incorporated by reference in its entirety.

### BACKGROUND OF THE INVENTION

### Field of the Invention

The present invention relates to a catalyst for the total hydrogenation of an olefin-containing hydrocarbon stream. The catalyst comprises a support material and platinum as sole catalytically active metal. The present invention also provides a process for the total hydrogenation of an olefin-containing hydrocarbon stream in at least one reactor with addition of hydrogen and using the catalyst of the invention.

### Description of Related Art

As is known, in hydrogenations, molecules having at least one double bond, for example olefins, undergo reaction through the addition of hydrogen to afford molecules having at least one fewer double bond. Corresponding processes have been known in the petrochemical industry for many years. Heterogeneous catalysts containing palladium are often used for this purpose and can achieve very good hydrogenation results. Palladium (Pd) is here usually the active metal that catalyses the actual hydrogenation reaction.

In recent years, the price of palladium has risen continuously. This continuous rise has led to a continuous rise too in the price of palladium-containing catalysts for use in hydrogenations. These catalysts are accordingly becoming increasingly unattractive from an economic perspective. As an alternative to palladium, various metals with hydrogenation activity are known, for example nickel. The problem here is that nickel catalysts are very difficult to handle in the hydrogenation of olefins.

### SUMMARY OF THE INVENTION

The object of the present invention was therefore to provide a catalyst for the total hydrogenation of an olefin-containing hydrocarbon stream that does not have the known problems. The catalyst should be of economic interest, but at the same time have high mass-specific activity.

Surprisingly, it has been found that mass-specific activity can be significantly increased by using platinum as the sole metal having hydrogenation activity. This makes it possible to use more cost-effective catalysts in the hydrogenation of olefins.

The present invention thus provides a heterogeneous catalyst for the total hydrogenation of an olefin-containing hydrocarbon stream, wherein the catalyst is a support material selected from the group consisting of aluminium oxide, silicon dioxide such as silica or silica gel, kieselguhr, aluminosilicates including zeolites, titanium dioxide, activated carbon and mixtures thereof and
comprises a sole catalytically active metal in an amount of from 0.05% to 1% by weight based on the total weight of the catalyst, wherein the catalytically active metal is platinum.

The present invention accordingly relates to a catalyst that comprises just one catalytically active metal, in this case platinum. Platinum is thus the sole metal having hydrogenation activity in the catalyst of the invention. The catalyst is thus not bimetallic, but monometallic. In particular, the catalyst of the invention is accordingly free of palladium or other metals such as tin, lead and nickel. Free in this context means that these metals are not present in the catalyst.

### BRIEF DESCRIPTION OF THE DRAWINGS

The FIGURE shows a preferred embodiment of the invention.

### DETAILED DESCRIPTION OF THE INVENTION

If the catalyst is present in the form of a granulate, the size of the particles (length of the longest edge) of the granulate is preferably in the range from 0.5 to 5 mm, preferably in the range from 1 to 4 mm. If the catalyst is present in the form of an extrudate, the size of the particles (length of the longest edge) of the extrudate is preferably in the range from 0.5 to 2.5 mm, preferably in the range from 1 to 2 mm. The particle size can be determined using known methods. The particles may also be spherical in shape, i.e. in the form of spheres. In this case, the diameter is preferably 0.5 to 5 mm, more preferably 2 to 4 mm.

The platinum serving as metal having hydrogenation activity is present in the catalyst particles of the catalyst of the invention preferably in an outer edge zone, where the edge zone thickness is less than 500 μm, preferably less than 250 μm. The metal can be deposited in the outer edge zone thermally or by chemical precipitation onto the support material. The edge zone can be determined by microscopic image analysis of cut catalyst particles. In this determination, the thickness of the edge zone can be measured using the software integrated into the microscope.

For catalysts intended to be used in small amounts (e.g. <3% by weight) in the hydrogenation of dienes or acetylene-containing components to olefins, preference is given to using supports having a high BET surface area, i.e. preferably supports having a BET surface area of >100 m²/g, more preferably supports having a BET surface area of >150 m²/g, in order to achieve the highest possible dispersion of the noble metal components. At higher concentrations of dienes or acetylene-containing components, preference is given to using supports having a small BET surface area, i.e. preferably supports having a BET surface area of <100 m²/g. When the BET surface area is low, the support material generally has low acidity, which means that the tendency to polymer formation on the support is reduced.

The present invention further provides a process for the total hydrogenation of an olefin-containing hydrocarbon stream, the process being carried out in a reactor with addition of hydrogen, characterized in that the process employs a heterogeneous catalyst that comprises a support material selected from the group consisting of aluminium oxide, silicon dioxide such as silica or silica gel, kieselguhr, aluminosilicates including zeolites, titanium dioxide, activated carbon or mixtures thereof, and platinum as catalytically active metal in an amount of from 0.05% to 1% by weight, preferably 0.1% to 0.3% by weight, in each case based on the total weight of the catalyst.

For the purposes of the present invention, total hydrogenation refers to a process in which all singly or polyunsaturated compounds (olefins, dienes, acetylene, etc.) are hydrogenated to saturated compounds, i.e. to the corresponding alkanes. In contrast, selective hydrogenation refers to the hydrogenation of individual molecules or of a group of molecules while at the same time obtaining other molecules or groups of molecules having a double bond. The present invention does not relate to the selective hydrogenation of just a portion of the olefins present. The aim of total hydrogenation is to hydrogenate as far as possible all the molecules in the olefin-containing hydrocarbon stream used that have a double bond, the hydrogenation thus taking place quantitatively. For technical reasons, a total hydrogenation is scarcely achievable or can be achieved only very laboriously. In accordance with the invention, the term total hydrogenation of an olefin-containing hydrocarbon stream thus means a process in which at least 99% of all olefins, preferably more than 99.5% of all olefins, in the olefin-containing hydrocarbon stream undergo hydrogenation.

According to a preferred embodiment, the process is a hydrogenation of olefin-containing hydrocarbon streams comprising linear or branched butenes (C4 streams), comprising linear or branched dibutenes (C8 streams), comprising linear or branched tributenes (C12 streams) or comprising linear or branched tetrabutenes (C16 streams). For the hydrogenation of these molecules it is typically not pure streams that are used, but industrially available mixtures (for example C4 cracking streams, raffinates or similar, and also mixtures from oligomerization processes) in which additional hydrocarbons are present alongside the substances to be hydrogenated.

In the total hydrogenation according to the invention, the hydrogen may be added in a stoichiometric amount or in a slight excess. In the total hydrogenation according to the invention, the hydrogen is metered in preferably in an amount of from 1.0 to 1.5 mol of hydrogen per mole of olefin, more preferably in an amount of from 1.0 to 1.3 mol of hydrogen per mole of olefin. Ideally, the hydrogen is added in an amount of from 1.0 to 1.1 mol of hydrogen per mole of olefin. Having the hydrogen present in not more than a slight excess is intended to prevent the occurrence of side reactions and/or reactions of the other hydrocarbons as a result of hydrogen oversupply.

The total hydrogenation can be carried out in a wide temperature range and is dependent on the length of the molecules, for example. The hydrogenation of the invention is preferably carried out at a temperature of between 2° and 300° C., more preferably between 25 and 250° C. and particularly preferably between 3° and 200° C.

In the total hydrogenation it is preferable for there to be at least a slight overpressure in order to promote the solubility of the hydrogen in the liquid phase. If the solubility of hydrogen in the liquid phase is low, a (very) high pressure may also be useful. The total hydrogenation of the invention can be carried out in the gas phase, in the liquid phase or in a mixed phase. Mixed phase in this context means that the hydrogen is not completely dissolved, but that in addition to the liquid phase a gaseous phase is also present. In a preferred embodiment of the present invention, the chosen reactor diameter should be such that the flow rate is sufficiently low that the liquid does not push the gas ahead of it, but gas and liquid are present together in the cross section. The Froude number of the liquid should be less than 0.3 here.

The hydrogenation of the present processes can be carried out in any suitable reactor, for example in a trickle-bed reactor, in a loop reactor with a downstream finisher reactor or in a pulse-flow reactor. Corresponding reactors are known to those skilled in the art. In a preferred embodiment of the present invention, the hydrogenation is carried out in at least two reactors, more preferably in at least one loop reactor and in at least one reactor operated in straight pass. Preferably, the loop reactor is the first reactor and the reactor operated in straight pass is the second reactor.

The hydrogenation is an exothermic reaction in which heat is evolved. This thermal energy would be destroyed by simple cooling. It is therefore preferable in accordance with the invention when the heat of reaction is used for thermal integration within the process. In a preferred embodiment of the present invention, the heat of reaction of the hydrogenation is used to preheat the employed reactant stream by setting the temperature of the circulation cooler to above 100° C., preferably above 140° C. and more preferably above 160° C., in order to generate hot water utilizable for thermal integration or heating steam of a suitable pressure level.

The reaction mixture obtained by the total hydrogenation can be subsequently processed by a thermal separation, in particular by distillation. Such processes are familiar to those skilled in the art.

A preferred embodiment of the present invention is shown in the FIGURE. This FIGURE is for illustration purposes and does not constitute a limitation of the subject matter of the invention. In the FIGURE, an olefin-containing hydrocarbon stream (feed) and hydrogen (H₂) are passed into a reactor (1) in which the catalyst of the invention is present. The reactor (1), which is operated in a loop, is where the hydrogenation takes place. A portion of the reactor discharge is returned to the inlet of the reactor (1) via the heat exchanger (2). The heat exchanger is in this case configured as a circulation cooler in the sense that the recycled stream is cooled in order to generate hot water utilizable for thermal integration or heating steam of a suitable pressure level. Another portion of the reactor discharge is supplied to the reactor (3), in which a catalyst of the invention is likewise present. In the reactor operated in straight pass (3), residual olefins are hydrogenated and a product stream (4) is obtained.

### EXAMPLES

Hydrogenation experiments were carried out in a reactor equivalent to reactor (1) (internal diameter 23 mm, height of active catalyst bed 41 cm) with loop and heat exchanger in the loop. In the experiments, a catalyst with platinum as active metal was used first. The catalyst containing platinum was then replaced with a catalyst containing palladium. Analogous hydrogenation experiments were carried out for comparison.

Hydrogenation experiments of analogous C12 olefins (molecules having 12 carbon atoms and one double bond per molecule) were carried out in a fixed-bed reactor (internal diameter 23 mm, height of active catalyst bed 41 cm) with additional circulation. In one experiment, a catalyst 1 with platinum (Pt) as active metal (0.5% by weight Pt on Al₂O₃—extrudates having a nominal size of 2 mm) was used and the comparative experiment used a catalyst 2 with palladium (Pd) as active metal (0.5% by weight Pd on Al₂O₃—analogous support to catalyst 1: Evonik Noblyst® H14271).

The experimental parameters achieved in the experiments are shown in Table 1 for catalyst 1 (Pt) and in Table 2 for catalyst 2 (Pd). The concentration of double bonds is obtainable via the reported bromine numbers (Br No.=grams of bromine consumed in a titration per 100 g of sample. For each mole of double bond, one mole of bromine is consumed.). The Br No. indicates the degree of hydrogenation. The lower the number, the fewer double bonds are still present. In addition, the tables state the resulting reaction constants=calculated rate constants of the hydrogenation reaction at the starting temperature of the active catalyst. The higher this value, the more rapidly the reaction proceeds and the higher the activity of the catalyst employed.

TABLE 1
Experimental parameters for hydrogenation with Pt catalyst

| | Experiment | Experiment |
|---|---|---|
| Catalyst 1 | 1 | 2 |
| Supply per unit amount of catalyst (kg/h*kg) | 5.4 | 5.5 |
| H₂ pressure (bar) | 13 | 12 |
| H₂ per unit amount of olefin (mol/h/mol/h) | 1.1 | 1.1 |
| Starting temperature of the active catalyst | 139 | 117 |
| Bromine number in the reaction product at | 0.15 | 0.91 |
| the reactor outlet | 12 | 8 |
| Reaction constant | | |

TABLE 2
Experimental parameters for hydrogenation with Pd catalyst

| | Experiment | Experiment |
|---|---|---|
| Catalyst 2 | 3 | 4 |
| Supply per unit amount of catalyst (kg/h*kg) | 6.3 | 6.0 |
| H₂ pressure (bar) | 14 | 13 |
| H₂ per unit amount of olefin (mol/h/mol/h) | 1.2 | 1.1 |
| Starting temperature of the active catalyst | 141 | 154 |
| Bromine number Br No. in the reaction | 9.25 | 0.98 |
| product at the reactor outlet | | |
| Reaction constant | 3 | 6 |

From the values reported in the two tables, it can be inferred that both the activity of the catalyst containing Pt and the activity of the catalyst containing Pd increase with increasing temperature. The calculated reaction constants in each case increase with temperature for each catalyst type. Astonishingly, it was also found that the activity of the catalyst containing platinum is already at a relatively low temperature higher than that of the equivalent catalyst containing palladium. Generally, the catalyst containing platinum has a higher activity than the catalyst containing palladium at the same temperature.

1. A heterogeneous catalyst for the total hydrogenation of an olefin-containing hydrocarbon stream, the catalyst comprising:
a support material selected from the group consisting of aluminium oxide, silicon dioxide, kieselguhr, aluminosilicates, titanium dioxide, activated carbon, and mixtures thereof and
a sole catalytically active metal in an amount of from 0.05% to 1% by weight based on a total weight of the catalyst, wherein the catalytically active metal is platinum.

2. The heterogeneous catalyst according to claim 1, wherein the catalyst contains platinum in an amount of from 0.1% to 0.3% by weight based on the total weight of the catalyst.

3. The heterogeneous catalyst according to claim 1, wherein the support material is selected from the group consisting of silicon dioxide, aluminium oxide, titanium dioxide, and mixtures thereof.

4. The heterogeneous catalyst according to claim 1, wherein the platinum is present in catalyst particles in an outer edge zone, a thickness of the edge zone being less than 500 μm.

5. A process, comprising:
hydrogenating, in a total hydrogenation, an olefin-containing hydrocarbon stream,
wherein at least 99% of all olefins in the olefin-containing hydrocarbon stream undergo hydrogenation,
wherein the process is carried out in a reactor with addition of hydrogen, and
wherein the process employs the heterogeneous catalyst according to claim 1.

6. The process according to claim 5, wherein the hydrogenation is carried out in a trickle-bed reactor, in a loop reactor with a downstream finisher reactor, or in a pulse-flow reactor.

7. The process according to claim 5, wherein a heat of reaction of the hydrogenation is used to preheat the reactants by setting a temperature of a circulation cooler to above 100° C., so as to generate hot water utilizable for thermal integration or heating steam of a suitable pressure level.

8. The process according to claim 5, wherein, in the total hydrogenation, the hydrogen is metered in an amount of from 1.0 to 1.5 mol of hydrogen per mole of olefin.

9. The process according to claim 5, wherein, in the total hydrogenation, the hydrogen is metered in an amount of from 1.0 to 1.3 mol of hydrogen per mole of olefin.

10. The process according to claim 5, wherein, in the total hydrogenation, the hydrogen is metered in an amount of from 1.0 to 1.1 mol of hydrogen per mole of olefin.

11. The process according to claim 5, wherein the olefin-containing hydrocarbon stream is at least one selected from the group consisting of olefin-containing hydrocarbon streams comprising linear or branched butenes, linear or branched dibutenes, linear or branched tributenes, and linear or branched tetrabutenes.

12. The process according to claim 5, wherein the total hydrogenation is carried out at a temperature of between 2° and 300° C.

13. The process according to claim 5, wherein, in the total hydrogenation, more than 99.5% of all olefins in the olefin-containing hydrocarbon stream undergo hydrogenation.

14. The heterogenous catalyst according to claim 1, wherein the support material is silicon dioxide, and wherein the silicon dioxide is silica or a silica gel.

15. The heterogeneous catalyst according to claim 1, wherein the support material is an aluminosilicate, and wherein the aluminosilicate is a zeolite.

16. The heterogeneous catalyst according to claim 4, wherein the thickness of the edge zone is less than 250 μm.

17. The process according to claim 7, wherein the temperature of the circulation cooler is above 160° C.

18. The process according to claim 7, wherein the temperature of the circulation cooler is above 140° C.
